(19) United States
(12) Patent Application Publication
(10) Pub. No.: US 2006/0178439 A1
(43) Pub. Date: Aug. 10, 2006

(54) Crystalline form of zoledronic acid

(76) Inventors: Pradeep Kumar Mohakhud, Hyderabad (IN); Veerender Murki, Secunderabad (IN); Kishore Babu Nandanmudi, Hyderabad (IN); Moses Babu, Secunderabad (IN); Surajit Banerjee, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/324,578

(22) Filed: Jan. 3, 2006

Related U.S. Application Data
(60) Provisional application No. 60/641,172, filed on Jan. 4, 2005.

(51) Int. Cl. A61K 31/12

(52) U.S. Cl. 514/678

(57) ABSTRACT

Zoledronic acid trihydrate.

### CROSS-REFERENCE TO RELATED APPLICATION

This application is a nonprovisional filing of copending U.S. Provisional Application No. 60/641,172 filed on Jan. 4, 2005, the entire content of which is incorporated herein by this reference.

### INTRODUCTION TO THE INVENTION

The present invention relates to a crystalline zoledronic acid trihydrate and a process for the preparation thereof.

Chemically zoledronic acid is (1-Hydroxy-2-imidazol-1-yl-phosphonoethyl) phosphonic acid and can be structurally represented by Formula I.

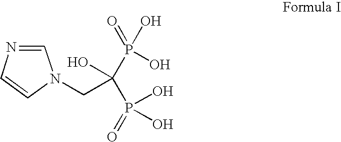

Zoledronic acid is a third generation bisphosphonate derivative characterized by a side chain that includes an imidazole ring. It inhibits osteoclast bone resorption and is used for the treatment of tumor-induced hypercalcemia. It is commercially available in products sold under the brand name ZOMETA™ in vials as a sterile powder or solution for intravenous infusion. Each vial contains 4 mg of zoledronic acid (anhydrous), corresponding to 4.264 mg of zoledronic acid monohydrate.

Chemical synthesis of zoledronic acid has to date been directed to the preparation of the monohydrate substance U.S. Pat. No. 4,939,130 discloses zoledronic acid and, in Example 10, a process for making zoledronic acid as shown in Scheme 1.

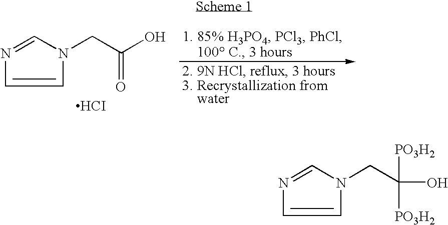

Briefly, the process comprises reacting 2-(1-imidazolyl) acetic acid hydrochloride with phosphoric acid in the presence of phosphorous trichloride and hydrochloric acid to yield zoledronic acid, which is precipitated by dilution with acetone. The crude zoledronic acid thus obtained is recrystallized in water. The final step of recrystallization of the crude substance from water provides the monohydrate of zoledronic acid.

International Application Publication No. WO 2005/063717 also involves a similar recrystallization from water in the final step providing the monohydrate compound of zoledronic acid.

International Application Publication No. WO 2005/005447 discloses various crystalline forms of zoledronic acid, its sodium salt and process for preparation thereof. It describes the preparation of crystalline Forms I, II, XII, and XVIII, which are monohydrates of zoledronic acid, and Forms XV, XX, and XXVI, which are anhydrous forms of zoledronic acid. It also describes various hydrated and anhydrous forms of the monosodium and disodium salts of zoledronic acid, and also describes amorphous zoledronate monosodium, disodium and trisodium salts.

Although a considerable amount of work has been done on the polymorphic characterization of zoledronic acid, there remains a need to identify other forms that can be generated by changing the reaction conditions.

As is well known in the art, the existence of polymorphic forms of any given compound cannot be predicted, and there is no standard procedure for proceeding to make a previously unknown polymorphic form. Even after a polymorph has been identified, there is no possibility of predicting whether any additional forms will ever be discovered. This situation has been the subject of recent articles, including A. Goho, “Tricky Business,” Science News, Vol. 166, No. 8, pages 122-123 (August 2004).

Moreover, regulatory authorities throughout the world require that all possible crystalline forms of the same active compound be synthesized and characterized as completely as possible. It is also required that the commercial product should not contain traces of any of the other forms or, if present, the percentages of each of the forms be well characterized to avoid changes in the dissolution and bioavailability characteristics of drug substance during storage.

There is thus a continuing need to prepare new polymorphic forms of pharmacologically active compounds of commercial interest such as zoledronic acid, which provide the pharmaceutical formulation scientist with a broader spectrum of crystalline forms of an active ingredient to choose from, based on their differing physiochemical properties.

### SUMMARY OF THE INVENTION

The present invention relates to a crystalline trihydrate of zoledronic acid and a process for its preparation.

One aspect of the invention provides a crystalline trihydrate of zoledronic acid characterized by its single crystal X-ray diffractogram (XRD), X-ray powder diffraction pattern (XRPD), infrared absorption spectrum (IR), and differential scanning calorimetry (DSC) curve.

In another aspect, the present invention provides a process for the preparation of the crystalline trihydrate of zoledronic acid comprising;
1) providing a solution of anhydrous zoledronic acid in a suitable solvent at elevated temperatures; and
2) subsequently cooling the solution to recover zoledronic acid trihydrate crystals.

A further aspect of the invention provides a process for the preparation of zoledronic acid monohydrate from crystalline zoledronic acid trihydrate.

### BRIEF DESCRIPTION OF THE DRAWINGS

FIG. 1 is an X-ray powder diffraction pattern of a crystalline trihydrate of zoledronic acid, where the vertical axis is intensity and the horizontal axis is the 2θ angle, in degrees.

FIG. 2 is an infrared absorption spectrum of a crystalline trihydrate of zoledronic acid, where the vertical axis is percent transmission and the horizontal axis is wavenumbers (cm⁻¹).

FIG. 3 is a differential scanning calorimetry curve of a crystalline trihydrate of zoledronic acid, where the vertical axis is milliwatts and the horizontal axis is temperature in ° C.

FIG. 4 is the single crystal structure of zoledronic acid trihydrate.

FIG. 5 is a simulated powder diffraction pattern from the single crystal data for a crystalline trihydrate of zoledronic acid, where the vertical axis is intensity and the horizontal axis is the 2θ angle, in degrees.

FIG. 6 shows a thermogravimetric analysis curve of a crystalline zoledronic acid trihydrate, superimposed on the differential scanning calorimetry curve for the compound.

### DETAILED DESCRIPTION

The present invention provides a crystalline trihydrate of zoledronic acid and a process for its preparation.

In one aspect of the invention, a crystalline trihydrate of zoledronic acid is provided which is characterized by any of its single crystal X-ray diffraction (“XRD”) parameters, X-ray powder diffraction (“XRPD”) pattern, infrared absorption (“IR”) spectrum, and differential scanning calorimetry (“DSC”) curve.

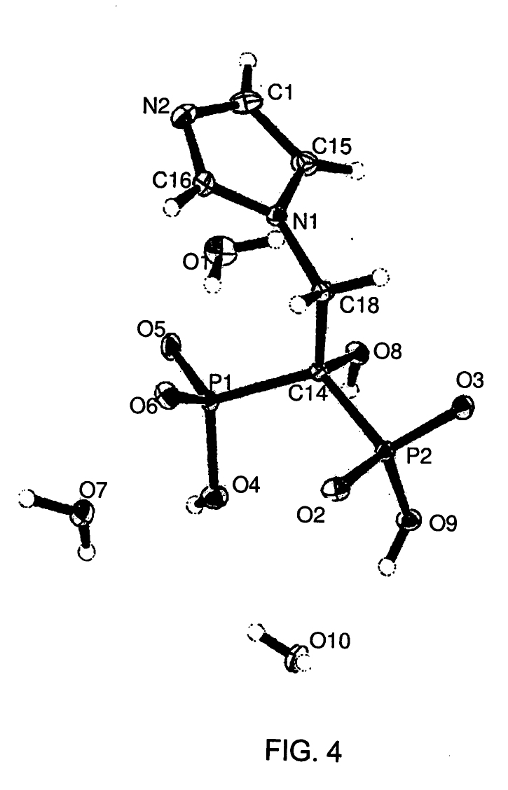

The crystal structure of zoledronic acid trihydrate is shown in FIG. 4. The trihydrate crystallizes in the triclinic space group P1 with the unit cell parameters as given in Table 1.

TABLE 1
Space group and unit cell parameters for zoledronic acid trihydrate.

| Parameter space group | Trihydrate |
|---|---|
| Cell dimensions | P2₁/c (No. 14) |
| 1a (Å) | 6.863 (2) |
| b (Å) | 9.439 (3) |
| c (Å) | 10.808 (3) |
| α (Å) | 65.175 (7) |
| β (Å) | 76.816 (11) |
| γ (Å) | 81.386 (13) |
| Volume (Å³) | 617.6 (3) |
| Z (Molecules/Unit cell) | 2 |

The packing in three dimensions is stabilized by strong intra- and inter-molecular hydrogen bonding as given in Table 2.

TABLE 2
Hydrogen bond parameters

| D-H . . . A | D-H . . . (Å) | H-A . . . (Å) | D-A . . . (Å) | D-H . . . A (°) | Symmetry codes |
|---|---|---|---|---|---|
| O1—H4 . . . O10 | 0.7900 | 2.0300 | 2.795(3) | 165.00 | 2 − x, 2 − y, −z |
| O9—H6 . . . O3 | 0.8100 | 1.8900 | 2.692(3) | 168.00 | 1 − x, 2 − y, 1 − z |
| O8—H8 . . . O6 | 0.9100 | 1.7000 | 2.611(3) | 178.00 | 2 − x, 1 − y, 1 − z |
| O9—H9 . . . O2 | 0.7600 | 1.9800 | 2.724(3) | 170.00 | 1 + x, y, z |
| O10—H10 . . . O2 | 0.8500 | 1.9500 | 2.783(3) | 167.00 | x, y, z |
| O10—H11 . . . 02 | 0.9100 | 2.4400 | 3.254(3) | 150.00 | 1 − x, 2 − y, −z |
| O8—H12---O9 | 0.7500 | 1.8300 | 2.575(3) | 173.00 | 2 − x, 2 − y, −z |
| O4—H13 . . . O3 | 0.8100 | 1.7900 | 2.599(3) | 175.00 | 1 − x, 2 − y, 1 − z |
| O7—H14 . . . O2 | 0.8100 | 1.8000 | 2.583(2) | 165.00 | 1 + x, y, z |

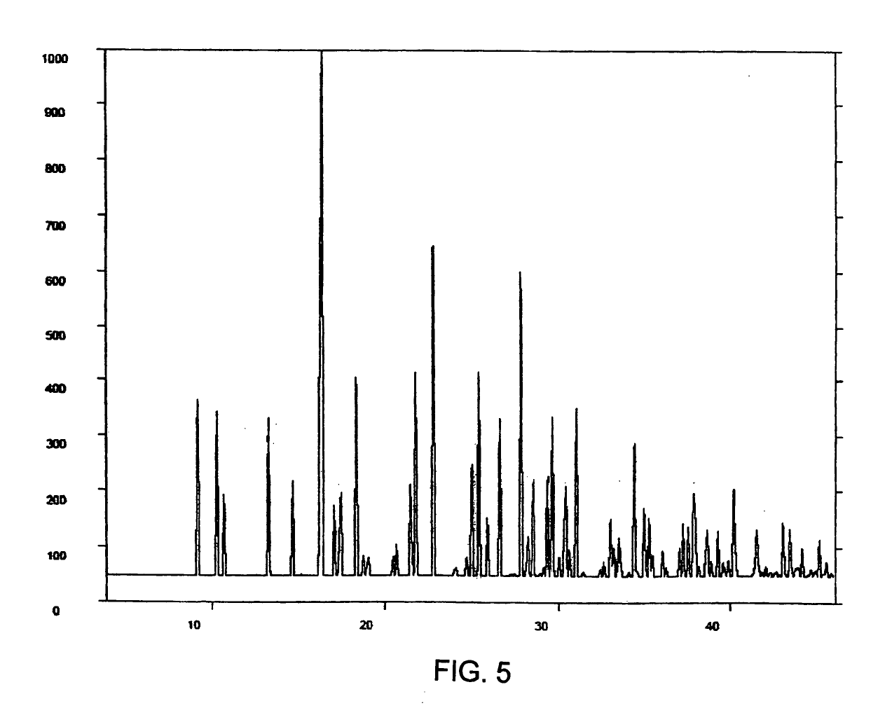

The XRD intensity data were collected on a Rigaku Mercury CCD area detector with graphite monochromatic Mo—Kα radiation. The structure was solved by direct methods and (SIR92) and refined by the least squares method. The present R factor is 0.038 and Rw=0.039 for 2110 observed reflection. The simulated powder diffraction pattern from single crystal data is shown in FIG. 5.

Zoledronic acid trihydrate is further characterized by its XRPD pattern, which differs from the other known forms. The XRPD data reported herein were obtained using Cu Kα radiation, having the wavelength 1.541 Å, and was measured on a Bruker Axe, D8 Advance Powder X-ray Diffractometer.

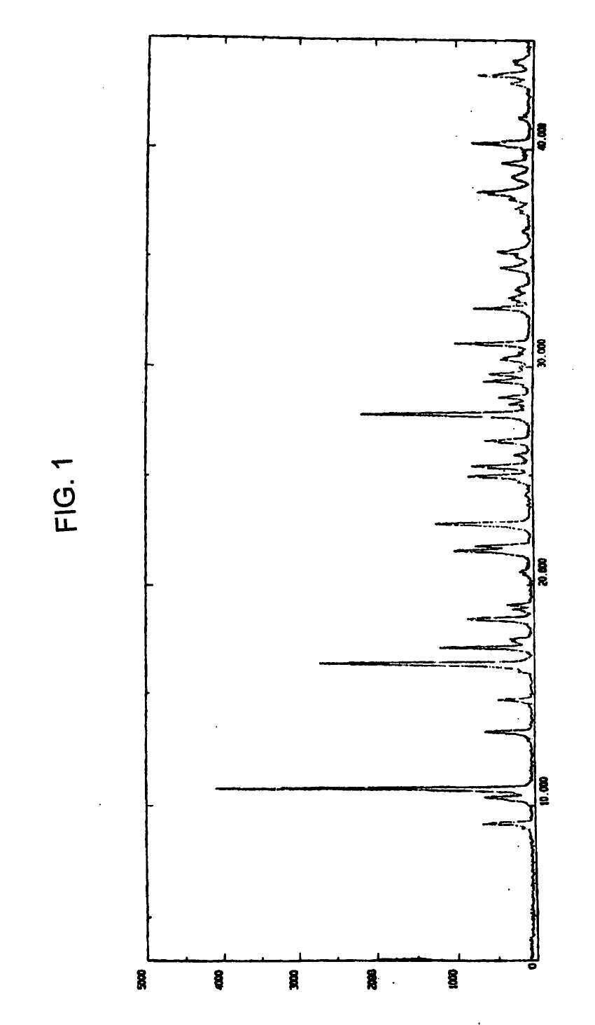

The crystalline trihydrate of zoledronic acid is characterized by its XRPD pattern substantially in accordance with the pattern of FIG. 1. The crystalline trihydrate of zoledronic acid is also characterized by an XRPD pattern having significant peaks at about 10.8, 16.4, 17.1, 18.4, 21.6, 24.9, 25.4, 27.8, 31.0, and 32.6, ±0.2 degrees 2θ. It is also characterized by the additional XRPD peaks at about 38.0, 40.2, 21.8, 9.2, 10.3, and 43.4, ±0.2 degrees 2θ.

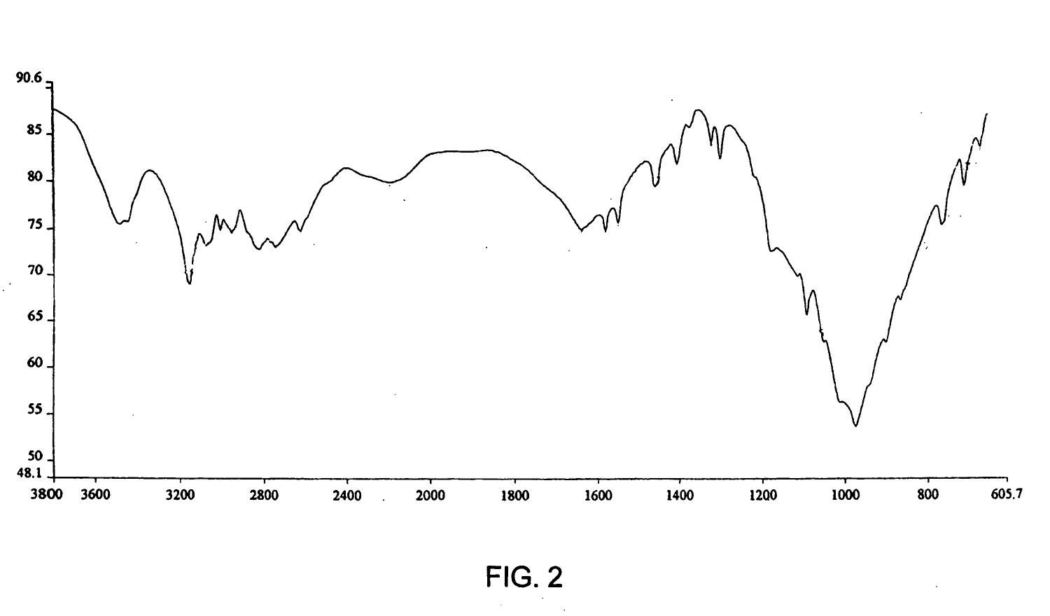

The crystalline trihydrate of zoledronic acid is also characterized by an infrared absorption spectrum in potassium bromide comprising peaks at about 671, 712, 766, 975, 1301, 1323, 1406, 1460, 1550, 2826, 3154, and 3484, +5 cm⁻¹. The crystalline trihydrate of zoledronic acid trihydrate is also characterized by its infrared absorption spectrum in potassium bromide substantially in accordance with the spectrum of FIG. 2.

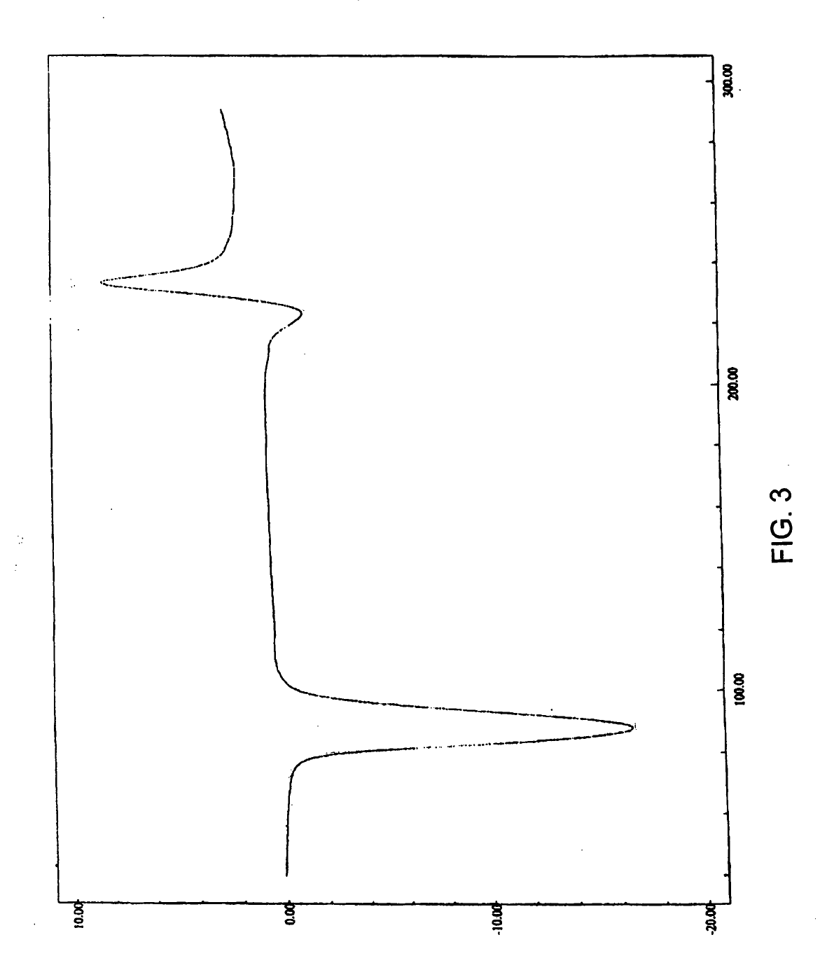

The crystalline trihydrate of zoledronic acid is also characterized by a differential scanning calorimetry curve substantially in accordance with the curve of FIG. 3. The crystalline trihydrate of zoledronic acid is also characterized by a DSC curve having an exotherm at about 234, and endotherms at about 224 and about 88° C.

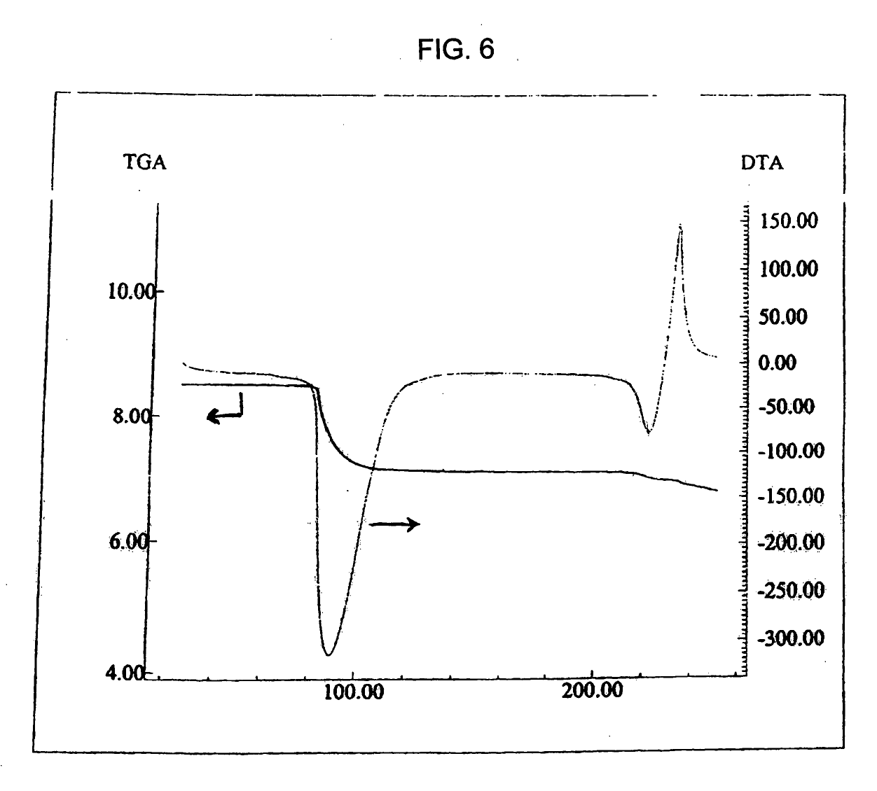

The crystalline trihydrate of zoledronic acid is also characterized by a thermogravimetric analysis curve substantially in accordance with the “DTA” curve of FIG. 6, showing the loss of three molecules of water. In FIG. 6, the left vertical axis is milligrams of sample, the right vertical axis is millivolts from a DSC thermocouple, and the horizontal axis is temperature, in ° C.

In another aspect, the present invention provides a process for the preparation of the crystalline trihydrate of zoledronic acid.

In an embodiment, a process for the preparation of trihydrate comprises dissolving zoledronic acid in a solvent or mixture of solvents at a suitable temperature, allowing the solution to cool and then recovering the separated zoledronic acid trihydrate crystals.

Suitable solvents useful in the preparation of the trihydrate of zoledronic acid include water alone or in combination with an organic solvent, such as for example alcohols such as methanol, ethanol, propanol, tertiary butanol, n-butanol; ketones like acetone, propanone; acetonitrile, dimethylformamide, dimethylsulphoxide, dioxane, and the like; and mixtures thereof.

In a related embodiment, the invention involves heating a solution of zoledronic acid in the solvent or mixture of solvents to a temperature of about 60 to 80° C., or about 70 to 75° C., to get a clear solution.

The temperature used for the dissolution of zoledronic acid determines the particular polymorphic form of zoledronic acid which will which will be ultimately obtained. Accordingly, when the solution is heated to higher temperatures of about 90 to 95° C., the crystalline monohydrate is produced and when lower temperatures, such as in the range of about 60 to 80° C., or about 70 to 75° C., are used, a trihydrate crystalline form is obtained.

The solution can be maintained in this temperature range for about 1 minute to any desired time. If the mixture is heated to about 75° C., the minimum required maintenance time at the elevated temperature, before cooling commences, is negligible.

An additional embodiment of the process involves a gradual cooling of the solution of zoledronic acid in the solvent or solvent mixture, to ambient temperatures. The time taken for the reaction mass to attain room temperature may range from about 1 to 2 hours, or about 1 to 5 hours, or about 1 to 20 hours, depending on the size of the batch being processed. There is no disadvantage to further extending the cooling period, other than an increased processing expense, and an appropriate time for a given batch size can be determined with little effort by one skilled in the art. The cooling of the solution may be achieved by simple radiation cooling under atmospheric conditions, accompanied by stirring, or through the use of controlled cooling mechanisms such as for example circulation of cooling media in jacket vessels and the like. Such techniques for gradual cooling are well known to a person skilled in the art and are all included herein without limitation.

The crystallization may be performed with stirring at ambient or reduced temperatures such as for example about 20° C. to about 25° C. or lower until the desired crystal yield has been obtained, such as for about one hour to about 72 hours. The crystallization step may further include facilitative measures known to one skilled in the art. For example, crystallization step may further include cooling the solution, heating the solution, or adding an agent to induce precipitation.

Recovery of the isolated solid can be performed by any means including, but not limited to, filtration, centrifugation, and decanting. The crystalline form may be recovered from any composition containing the crystalline form and the solvent or solvents including but not limited to a suspension, solution, slurry, and emulsion.

The obtained compound can be further dried under ambient or reduced pressure. For example, drying can be performed under reduced pressure or under atmospheric pressure at a temperature of at about 40° C. to 60° C., or 70° C. to 80° C., or higher. Drying can be performed until a desired residual solvent content has been obtained, such as for a duration of about 2 hours to 24 hours, or about 3 to 6 hours.

Yet another aspect of the invention provides a process for the preparation of zoledronic acid monohydrate from crystalline zoledronic acid trihydrate.

The process for the conversion involves any one of the processes of extended drying of the trihydrate at temperatures higher than 50° C. under vacuum, or slurrying of the trihydrate in an organic solvent, or by a solvent-antisolvent technique.

In one embodiment, the conversion of zoledronic acid trihydrate to zoledronic acid monohydrate may be performed by drying the compound for an extended time. In some instances, the duration of drying may range from about 5 hours, about 10 hours, about 15 hours, or about 20 hours or more. The time required will depend on the temperatures and other environmental conditions used, and can easily be determined by simple experimentation.

The temperatures for drying may range from 40 to 90° C., or 60 to 70° C., or 55 to 60° C., and the compound may be dried under ambient or reduced pressure. For example, drying can be performed under reduced pressure or under atmospheric pressure in any one of an air oven, vacuum oven, or tray drying and the like can be used. Optionally, drying can be conducted under an inert atmosphere.

In an embodiment, conversion of zoledronic acid trihydrate to zoledronic acid monohydrate may be accomplished by slurrying in a suitable solvent.

Suitable solvents which can be used for slurrying are ketones like acetone, ethyl methyl ketone, propanone, and the like.

The slurrying may be accompanied by stirring or slurrying may be performed simply by keeping the mixture static for a period of time.

In another embodiment, the invention provides a process for the preparation of zoledronic acid monohydrate involving recrystallization by a solvent-antisolvent technique.

The process comprises providing zoledronic acid and a suitable solvent, and heating the mixture to provide a clear solution followed by addition of an antisolvent to obtain a precipitate of the required product.

Any form of zoledronic acid may be used in the preparation of the solution in the solvent or mixture of solvents such as for example zoledronic acid trihydrate or other crystalline or amorphous forms of zoledronic acid including any of its salts, solvates, or hydrates.

Suitable solvents which can be used for dissolution include for example: water; alcohols such as methanol, ethanol, propanol, n-butanol; dimethylformamide; dimethylsulphoxide; tetrahydrofuran; and the like, and mixtures thereof.

Antisolvents which can be used include for example: hydrocarbons such as n-hexane, n-heptane, and toluene; ketones such as acetone, propanone, ethyl methyl ketone, and butanone; ethers such as diethyl ether, isopropyl ether, etc; esters such as ethyl acetate, tertiary butyl acetate and the like; halogenated hydrocarbons such as dichloromethane, 1,2-dichloroethane, chloroform, carbon tetrachloride; and mixtures thereof.

The dissolution procedure can be carried out at elevated temperatures ranging from about 95 to 120° C. Heating may be accompanied by stirring or agitation occasionally by any means including but not limited to mechanical and magnetic means. The amount of solvent should be sufficient to dissolve the zoledronic acid to form a concentrated solution.

Addition of anti-solvent to the solution of the zoledronic acid may be carried out at elevated temperatures of about 90 to 120° C., or 60 to 90° C., or at ambient temperatures, or at lower temperatures ranging from about 0 to 15° C.

Recovery of the isolated solid can be performed by any means including but not limited to filtration, centrifugation, and decanting. The crystalline form may be recovered from any composition containing the crystalline form and the solvent or solvents including but not limited to a suspension, solution, slurry, and emulsion.

The obtained compound can be further dried under ambient or reduced pressure. For example, drying can be performed under reduced pressure or under atmospheric pressure at a temperature of at about 40° C. to 60° C., or 70° C. to 80° C., or higher. Drying can be performed for a duration of about 2 hours, or about 5 hours or more, depending on the amount of residual solvent content that is aceptable.

Certain aspects and embodiments of the invention are further illustrated by the following examples, which should not be construed as limiting the scope of the invention.

### EXAMPLE 1

Preparation of Zoledronic Acid Trihydrate

5 g of anhydrous zoledronic acid was taken into a round bottomed flask equipped with a magnetic stirrer, condenser and oil bath, then 150 ml of water was added to it. The reaction mass was heated slowly to 73° C. to obtain a clear solution. The solution was filtered while hot to make it particle free. The clear filtrate was taken into a fresh round-bottomed flask and allowed to cool to 30° C. The reaction mass was stirred at 30° C. for 10 minutes. The separated solid was filtered under vacuum. The compound was suction dried under a vacuum of 600 mm Hg for 10 minutes to get 3.6 g of the title compound.

Samples of this product were analyzed, to generate all of FIGS. 1-6.

Moisture content: 15.5% (w/w).

Melting point: 238±3° C.

### EXAMPLE 2

Conversion of Trihydrate to Monohydrate by Drying

1 g of zoledronic acid trihydrate was taken in a clean Petri dish. The compound was then dried in a vacuum oven at 60° C. under a vacuum of 600 mm Hg for 16 hours to obtain zoledronic acid monohydrate.

### EXAMPLE 3

Conversion of Trihydrate to Monohydrate by Slurrying

5 ml of acetone was placed into a round bottom flask along with 0.5 g of zoledronic acid trihydrate. The mixture was then stirred at 28° C. for 30 minutes. The mixture was filtered under a vacuum of 600 mm Hg and the solid was finally dried under vacuum at 28° C. to give the monohydrate of zoledronic acid.

### EXAMPLE 4

Conversion of Trihydrate to Monohydrate Using Solvent-Antisolvent Technique

30 ml of water was placed into a round bottomed flask along with 1 g of zoledronic acid trihydrate. The mixture was stirred for about 10 to 20 minutes at 28° C. followed by heating to 99° C. and was maintained at 99° C. for another 15 minutes. The mass was then allowed to cool by radiation to 67° C. At this temperature 10 ml of methanol was added to precipitate the product, and the mass was then stirred until it had cooled to 28° C. The separated solid was filtered under vacuum and was washed with 10 ml of water. The solid was than suction dried under a vacuum of 600 mm Hg for 30 minutes at 28° C. and finally dried at 59° C. under a vacuum of 600 mm Hg for 12 hours to afford the crystalline monohydrate of zoledronic acid.

### EXAMPLE 5

Conversion of Trihydrate to Monohydrate Using Solvent-Antisolvent Technique

30 ml of water was placed into a round bottom flask along with 1 g of zoledronic acid trihydrate. The mixture was stirred for about 10 minutes at 28° C. followed by heating to 99° C. and was maintained at 99° C. for another 30 minutes. The mixture was then allowed to cool by radiation to 57° C. At this temperature, 10 ml of acetone was added to precipitate the product. The mixture was then stirred until it had cooled to 28° C. The mass was maintained at 28° C. for 3 hours. The separated solid was then filtered under a vacuum of 600 mm Hg. The solid was suction dried for 45 minutes and finally dried under vacuum of 600 mm Hg at 60° C. for about 3 hours to afford the crystalline monohydrate of zoledronic acid.

We claim:

1. Zoledronic acid trihydrate.

2. The zoledronic acid trihydrate of claim 1, having an X-ray powder diffraction pattern using Cu Kα radiation substantially in accordance with FIG. 1.

3. The zoledronic acid trihydrate of claim 1, having an X-ray powder diffraction pattern using Cu Kα radiation comprising peaks at about 10.8, 16.4, 17.1, 18.4, 21.6, 24.9, 25.4, 27.8, 31.0, and 32.6, ±0.2 degrees 20.

4. The zoledronic acid trihydrate of claim 2, having an X-ray powder diffraction pattern using Cu Kα radiation further comprising peaks at about 38.0, 40.2, 21.8, 9.2, 10.3, and 43.4, ±0.2 degrees 20.

5. The zoledronic acid trihydrate of claim 1, having an infrared absorption spectrum substantially in accordance with FIG. 2.

6. The zoledronic acid trihydrate of claim 1, having an infrared absorption spectrum comprising peaks at about 671, 712, 766, 975, 1301, 1323, 1406, 1460, 1550, 2826, 3154, and 3484, ±5 cm⁻¹.

7. The zoledronic acid trihydrate of claim 1, having a differential scanning calorimetry curve substantially in accordance with FIG. 3.

8. The zoledronic acid trihydrate of claim 1, having a differential scanning calorimetry curve comprising an exotherm at about 234° C., and endotherms at about 224° C. and about 88° C.

9. A process for preparing zoledronic acid trihydrate, comprising providing a solution of zoledronic acid in a solvent comprising water at temperatures of about 60 to 80° C., and cooling the solution to crystallize zoledronic acid trihydrate.

10. The process of claim 9, wherein a solution of zoledronic acid is at temperatures of about 70 to 75° C.

11. The process of claim 9, wherein a solvent comprises water and an organic solvent.

12. A process for converting zoledronic acid trihydrate to zoledronic acid monohydrate, comprising drying zoledronic acid trihydrate at temperatures about 40 to 90° C.

13. A process for converting zoledronic acid trihydrate to zoledronic acid monohydrate, comprising slurrying zoledronic acid trihydrate in a ketone.

14. A process for preparing zoledronic acid monohydrate, comprising providing an aqueous solution of zoledronic acid and adding an antisolvent for zoledronic acid.

15. The process of claim 14, wherein an antisolvent comprises a ketone.
